(19) United States
(12) Patent Application Publication
(10) Pub. No.: US 2003/0031689 A1
(43) Pub. Date: Feb. 13, 2003

(54) Method of skin exfoliation

(76) Inventors: Thomas Mammone, Farmingdale, NY (US); Donald F. Collins, Plainview, NY (US); David C. Gan, Huntington Station, NY (US)

Correspondence Address: Karen A. Lowney, Esq., ESTEE LAUDER COMPANIES, 125 Pinelawn Road, Melville, NJ, 11747, US

(21) Appl. No.: 09/925,333

(22) Filed: Aug. 9, 2001

(51) Int. Cl. A61K 7/48; A61K 31/7024

(52) U.S. Cl. 424/401; 514/23

(57) ABSTRACT

The invention relates to a method for the exfoliation of skin, comprising applying to the skin a composition containing an effective amount of a phosphosugar. The invention also relates to a method for increasing glycosaminoglycan levels in skin, comprising applying to the skin a composition containing an effective amount of a phosphosugar. In a preferred embodiment, the phosphosugar is mannose-6-phosphate.

### FIELD OF THE INVENTION

The invention relates to a method for improving skin condition. More specifically, the invention relates to a method for improving the texture and surface of skin by enhancing the exfoliation process.

### BACKGROUND OF THE INVENTION

The stratum corneum represents the major chemical and physical barrier between the body and the environment. It is formed by a process in the epidermis which involves the transformation of germinative cells into terminally differentiated cells; the process of transformation takes approximately one month, by which time the terminally differentiated cells are shed from the skin surface. The cells at the outermost layer of the skin, which are constantly being sloughed off, are replaced by cells that are generated by the mitotic activity of the basal layer of the epidermis. In the course of their migration from the basal layers to the upper levels of the skin, these cells produce and accumulate keratin, to the point at which there is virtually no cytoplasm remaining; the cell then dies and is shed, to be followed by another phalanx of migrating epidermal cells.

In a perfect situation, this programmed migration and sloughing of cells from the surface of the skin leaves the skin always looking smooth and fresh. However, as any woman of a certain age knows, this system does not always perform perfectly, and when the exfoliation process is not performing under optimum circumstances, the accumulation of dead cells at the skin surface can result in a dull, patchy, irregular feel and appearance of the skin, which is of course particularly noticeable on the skin of the face. In recent years, the importance of supplementing the natural exfoliation process has become apparent, and numerous cosmetic and pharmaceutical materials have been promoted for this use. Retinoids, particularly retinoic acid, are frequently used to remove outer skin layers, to leave a fresh new layer of skin visible at the surface. Alpha hydroxy acids have also been widely used as exfoliation enhancers. Each of these materials can be very effective in promoting exfoliation, and are very popular products for a broad spectrum of consumers. However, for some potential users, the acidic nature of many of these materials can be irritating to sensitive skin, and certain of these, particularly retinoic acid, can render the skin very sensitive to sunlight, requiring the user to limit sun exposure.

The availability of a gentle, non-acidic exfoliant would provide a wider range of consumers with the ability to supplement the natural sloughing process, without the possibility of skin irritation. The present invention provides such an exfoliant.

### SUMMARY OF THE INVENTION

The present invention relates to a method of exfoliating skin which comprises applying to the skin an exfoliant-effective amount of a phosphosugar. Particularly preferred is a mannose phosphate, particularly mannose-6-phosphate. The method provides a gentle but effective means for sloughing off the dead outer layers of the skin, substantially without irritation to the user. The present method provides a level of exfoliation that can exceed that of many of the currently used products, such as alpha hydroxyacids, lactobionic acids, or N-acetyl glucosamine.

The invention also relates to a method of enhancing the synthesis of glycosaminoglycans, the main water-binding materials in skin by applying to the skin an effective amount of a phosphosugar. Enhanced synthesis of glycosaminoglycans results in increased water retention in the skin, and therefore, skin plumping and concurrent reduction in the appearance of lines and wrinkles in the skin.

### DETAILED DESCRIPTION OF THE INVENTION

It has been unexpectedly discovered that phosphosugars, i.e., phosphoric acid esters of sugars, are capable of enhancing the natural process of desquamation of the skin. The phosphosugars are naturally occurring sugars in the human body, and have relatively little potential for inducing irritation in a user, unlike many other currently popular exfoliants. Certain phosphosugars have previously been used in skin-related applications; for example, mannose phosphates have been shown to promote wound-healing activities. However, to Applicants' knowledge, they have not previously been used in cosmetic methods for enhancing exfoliation of the stratum corneum.

The phosphosugars useful in the present invention include, but are not limited to glucose-1- or -6-phosphate, mannose-6-phosphate, mannose-1-phosphate, galactose-6-phosphate, fructose-6-phosphate, glucose-1,6-diphosphate, or fructose-1,6-diphosphate. Particularly preferred in the method of the invention is a mannose phosphate, most preferably mannose-6-phosphate. Also included within the definition of phosphosugars for use in the present invention are the pharmaceutically and cosmetically acceptable salts thereof, e.g., mono- or disodium salts, as well as any precursor forms that when applied to the skin release the phosphosugar.

For use in the method of the invention, the phosphosugars can be combined with any pharmaceutically or cosmetically acceptable carrier, and applied in any form that is normally used on the skin. The term “pharmaceutically or cosmetically acceptable carrier” refers to a vehicle, for either pharmaceutical or cosmetic use, which vehicle delivers the active components to the intended target and which will not cause harm to humans or other recipient organisms. As used herein, “pharmaceutical” or “cosmetic” will be understood to encompass both human and animal pharmaceuticals or cosmetics, with which the active component is compatible, e.g., a gel, a cream, a lotion, an ointment, a mousse, a spray, a solid stick, a powder, a suspension, a dispersion, and the like. Techniques for formulation of various types of vehicles are well known to those skilled in the art, and can be found, for example, in Chemistry and Technology of the Cosmetics and Toiletries Industry, Williams and Schmitt, eds., Blackie Academic and Professional, Second Edition, 1996, and Remington's Pharmaceutical Sciences, 18th Edition, 1990, the contents of which are incorporated herein by reference. The formulations employed can also include other cosmetic or pharmaceutical ingredients, e.g., moisturizers, humectants, antiinflammatories, antioxidants, and the like. The effective amount of phosphosugar is defined as that amount which will reduce skin flakiness at least about 10% relative to a placebo, preferably at least about 20%. The actual amount will vary depending on the potency of the sugar employed; however, generally the amount used will be in the range of from about 0.01 to about 10%, preferably from about 0.1-5%, most preferably about 0.5-3%, by weight of the total composition. The most preferred sugar for use in the invention is mannose-6-phosphate.

Surprisingly, certain of the phosphosugars outperform other well-known and highly effective desquamation agents. In tests conducted to compare the efficacy of various agents of this type, mannose-6-phosphate caused a reduction in skin flakiness that exceeded the reduction achieved by N-acetyl glucosamine, lactobionic acid, and various mixtures of alpha hydroxy acids, all used at the same or higher levels than the mannose-6-phosphate. In a representative test, mannose-6-phosphate used at 1% reduced skin flakiness, a common measure of exfoliation efficacy, at a level of 24% after two weeks of treatment and 36% at four weeks. Comparable numbers for other exfoliants are 16% and 15% for 1% glucosamine, 16% and 25% for 2% lactobionic acid, and 27 and 25% for an approximately 2% mixture of alpha hydroxy acids. Thus, mannose-6-phosphate is particularly and unexpectedly effective for desquamation.

Formulations containing the phosphosugars of the invention may be used for exfoliation in the same manner recommended for any such products. In particular, the formulation may be applied on an as-needed basis, to “resurface” skin that is temporarily afflicted with a patchy, flaky or irregular texture. In many cases, however, application of the formulation will be chronic, to remedy a long-term reduction in the natural exfoliation process, by regular application of a phosphosugar. It is suggested as an example that topical application of the composition, in an amount of from about 0.1 mg/cm² to 2 mg/cm² of skin, be performed from about once per week to about 4 or 5 times daily, preferably from about 3 times a week to about 3 times daily, most preferably about once or twice per day. By “chronic” application, it is meant herein that the period of topical application may be over the lifetime of the user, preferably for a period of at least about one month, more preferably from about three months to about twenty years, more preferably from about six months to about ten years, more preferably still from about one year to about five years, thereby enhancing the process of desquamation.

In addition to the utility in exfoliation, it has also been determined that phosphosugars, particularly mannose-6-phosphate, can be used to enhance levels of glycosaminoglycans in skin cells. Glycosaminoglycans are a crucial component of connective tissue, and constitute a large proportion of the materials found in the intercellular spaces in the stratum corneum of the skin, as well as other areas of the body. The compounds constituting this group include hyaluronic acid, chondroitin sulfate, and heparan sulfate; these compounds, which are synthesized by skin cells, have a strong affinity for water, and play a large role in maintaining proper water levels in the skin. Thus, maintenance of high levels of glycosaminoglycans can be crucial to maintaining a healthy, properly moisturized skin. Without the proper level of water, the stratum corneum becomes inflexible and subject to cracking, thereby allowing further moisture to escape the skin, resulting in a variety of conditions related to the drying of the skin. In addition, the retention of water in the skin allows the skin to remain plumped, reducing the appearance of any lines or wrinkles that may be present. Thus, the ability of phosphosugars to enhance levels of glycosaminoglycans can improve all skin conditions that may be associated with lowered levels of glycosaminoglycans, such as dry skin, the appearance of lines and wrinkles, and other symptoms of chrono- or photoaging. The formulation and method of application of the sugar is, in general terms, similar to that described above for exfoliation. Effective amounts for increasing glycosaminoglycan levels are in the same broad ranges as for exfoliation, with the most preferred range being 0.01 to about 1%.

The invention will be further elucidated by reference to the following non-limiting examples.

### EXAMPLES

I. Use of Phosphosugar for Exfoliation

Thirty female subjects between the ages of 21 and 65 are selected to test the efficacy of mannose-6-phosphate in reducing skin flakiness, an indicator of exfoliation efficacy. The subjects are instructed not to use moisturizers or any other products on their hands, and their baseline measurements are taken. They are then randomly assigned to one of two treatment groups, and given the treatment product to self-administer to the right hand only, twice a day, in the morning after washing and in the evening at least 15 minutes before bedtime for four weeks. The left hand serves as the untreated control site. At the end of two and four weeks the subjects return for testing without applying the product for at least 12 hours and they are re-evaluated under the same conditions. One treatment group is given a placebo cosmetic base without active ingredients, and the other group is given the same base containing 1% mannose-6-phosphate.

To evaluate the efficacy of the treatment products, four D-Squame discs are firmly and evenly pressed on the back of each hand with a hand held uniform pressure device and removed by gently pulling away from the skin. The D-Squame discs are mounted on clear microscope slides and labeled according to panelist name and visit. Desquamation is evaluated from the D-Squame discs via an OPTIMA image analyzer. The measurements are timed as indicated above.

The D-Squame samples containing stratum corneocytes(i.e., skin flakes) are place under a camera on top of a light table and each image is imported into the image analyzer. The average Gray Value corresponding to the sample density is measured. The denser the sample, the higher the Gray Value difference. The greater the difference in % change from baseline between treated and untreated sites, the greater the reduction in skin flakiness, and therefore the greater the efficacy in desquamation. The results obtained indicate that the mannose-6-phosphate containing composition significantly reduced skin flakiness by about 24% at 2 weeks and about 30% at 4 weeks, whereas the placebo had substantially no effect on flakiness(reduction of 1-2%).

II. Use of Phosphosugar to Increase Glycosaminoglycans

Both mannose and mannose-6-phosphate are tested at various levels from 0.01 mg/ml to 1 mg/ml for their ability to increase the amount of glycosaminoglycans in Normal Human Dermal Fibroblasts(NHDF), using TGF- at 5 and 10 ng/ml as a positive control. NHDF cells are seeded and grown to confluence in a 24 well plate prior to being treated with the test extracts(n=3). At the same time as the treatment, the cultures are labeled with 1 Ci/ml of ³H-glucosamine. The cultures are incubated for 48 hours and extensively washed to remove unbound ³H-glucosamine prior to being lysed and counted on a scintillation counter. The resulting counts represent newly synthesized glycosaminoglycans. Since glucosamine is required for glycosaminoglycan synthesis, the radioactive glucosamine added to the culture will be incorporated into any glycosaminoglycans synthesized after the addition of the treatment samples. Relative protein levels are determined by comparing the absorbance at 280 nm, of the cell lysates, to the absorbances of a bovine serum albumin curve.

Results obtained show that mannose-6-phosphate at a level of 0.1 mg/ml increased glycosaminoglycan levels in NHDF cultures by 20%(when normalized to protein levels), while lower and higher levels did not have any significant effect. TGF-, the positive control, is found to increase levels by 28% at 10 ng/ml. Mannose has no significant effect on glycosaminoglycans.

What we claim is:

1. A method of exfoliating the skin comprising applying to the skin a composition containing an effective amount of at least one phosphosugar.

2. The method of claim 1 in which the phosphosugar is selected from the group consisting of glucose-1- or -6-phosphate, mannose-6-phosphate, mannose-1-phosphate, galactose-6-phosphate, fructose-6-phosphate, glucose-1,6-diphosphate, and fructose-1,6-diphosphate.

3. The method of claim 1 in which the phosphosugar is mannose-6-phosphate or mannose-1-phosphate.

4. The method of claim 1 in which the phosphosugar is mannose-6-phosphate.

5. The method of claim 1 in which the effective amount is from about 0.01 to about 10% by weight of the total composition.

6. The method of claim 4 in which the effective amount is from about 0.5 to about 3% by weight of the total composition.

7. A method for increasing levels of glycosaminoglycans in skin comprising applying to the skin a composition containing an effective amount of at least one phosphosugar.

8. The method of claim 7 in which the phosphosugar is selected from the group consisting of glucose-1- or -6-phosphate, mannose-6-phosphate, mannose-1-phosphate, galactose-6-phosphate, fructose-6-phosphate, glucose-1,6-diphosphate, and fructose-1,6-diphosphate.

9. The method of claim 7 in which the phosphosugar is mannose-6-phosphate or mannose-1-phosphate.

10. The method of claim 7 in which the phosphosugar is mannose-6-phosphate.

11. The method of claim 7 in which the effective amount is from about 0.01 to about 10% by weight of the total composition.

12. The method of claim 10 in which the effective amount is from about 0.5 to about 3% by weight of the total composition.

13. A method of treating a skin condition associated with a reduced level of glycosaminoglycans in the skin comprising applying to the skin a composition containing an effective amount of at least one phosphosugar.

14. The method of claim 13 in which the condition is selected from the group consisting of dry skin, lines and wrinkles, and the symptoms of chrono- and photoaging.

15. The method of claim 13 in which the phosphosugar is selected from the group consisting of glucose-1- or -6-phosphate, mannose-6-phosphate, mannose-1-phosphate, galactose-6-phosphate, fructose-6-phosphate, glucose-1,6-diphosphate, and fructose-1,6-diphosphate.

16. The method of claim 13 in which the phosphosugar is mannose-6-phosphate or mannose-1-phosphate.

17. The method of claim 13 in which the phosphosugar is mannose-6-phosphate.

18. The method of claim 13 in which the effective amount is from about 0.01 to about 10% by weight of the total composition.

19. The method of claim 17 in which the effective amount is from about 0.01 to about 1% by weight of the total composition.
